(19) United States
(12) Patent Application Publication
(10) Pub. No.: US 2022/0062019 A1
(43) Pub. Date: Mar. 3, 2022

(54) Wearable Device for Posture Correction and Muscle Training

(71) Applicant: Ji XIAO, Alhambra, CA (US)

(72) Inventor: Ji XIAO, Alhambra, CA (US)

(21) Appl. No.: 17/002,770

(22) Filed: Aug. 25, 2020

(51) Int. Cl. A61F 5/02; A61F 5/37

(52) U.S. Cl. CPC A61F 5/026; A61F 5/3761

(57) ABSTRACT

A wearable device, for posture correction and muscle training, includes a wearable assembly and two resilience supporters, wherein the resilience supporter has an resilience tendency to be upright, wherein the wearable assembly is detachably arranged on the resilience supporter, wherein the wearable assembly is supported by the resilience supporter and is moved along with the resilience supporter, so that the user's body fits the wearable assembly and applies an force against the resilience supporter, wherein the wearable device has no need to restrain the user's body, and can provide portable posture correction to ensure long-term major muscle activity in daily life.

### NOTICE OF COPYRIGHT

A portion of the disclosure of this patent document contains material which is subject to copyright protection. The copyright owner has no objection to any reproduction by anyone of the patent disclosure, as it appears in the United States Patent and Trademark Office patent files or records, but otherwise reserves all copyright rights whatsoever.

### BACKGROUND OF THE PRESENT INVENTION

### Field of Invention

The present invention relates to wearable exercise device, and more particularly to a wearable device for spine posture correction and main muscle group training.

### Description of Related Arts

With the increase of science and technology, daily work is increasingly lacking in physical labor. More and more users need to make plans of regular exercise and fitness, so as to keep healthy in body.

For conventional exercise device, a certain amount of weight is generally required to activate muscles or to stretch the body posture. Then, the conventional exercise device is usually used in a specific room and cannot be used anytime and anywhere.

However, in daily life, users do not have the opportunity to use conventional exercise device for a long time. Most of the time, the user's body is in an inactive state, such as in a sitting posture for work. Even while in standing or walking posture, the muscles and bones of the body are relaxed due to head down working or the use of smart phones, and the major muscle groups are not activated, especially the chest, abdomen, and back muscle groups are not fully activated. If these major muscle groups are not used for a long time, not only will the muscles become smaller and atrophy, but the bones will also be distorted and deformed. As the work style has gradually changed from manual work to mental work, the aging and deformation of muscles and bones have gradually advanced, and many young people have already experienced symptoms such as partial muscle atrophy.

For these symptoms, prevention should be given priority. Some conventional elastic restraint belts and braces for posture correction are actually one step late. Moreover, the main principle of these conventional restraint belts is to limit the bending of the body. If the posture is not maintained in the manner of restraint belts, it will cause unbearable pain. Once the elasticity of the belts disappears, it has no effect. The restraint belt may not be suitable for every user. Improper use may cause lumbar lordosis and thoracic kyphosis, which is counterproductive. Some restraint belts focus on the lumbar spine, which may be harmful to the internal organs of the waist while restraining too much tight.

A more effective way to correct the posture is to exercise or do fitness at ordinary times. Through the persistent activation of the major muscle groups, the activity of the muscles is maintained, so that the body posture is naturally maintained. In other words, even if it is sitting, standing, or walking, the major muscle groups need to be confronted with strength to avoid prolonged laziness. Conventional exercise device that can be used daily is for activating small muscle groups, such as a gripper or an arm bar. These device have little effect on core strength in muscle training.

It is needed by the market to provide wearable devices for spine posture correction and major muscle group exercise anytime and anywhere in daily life.

### SUMMARY OF THE PRESENT INVENTION

The invention is advantageous in that it provides a wearable device for posture correction and muscle training, which has not need to tighten or restraint the user's body, can provide portable posture correction, and ensure long-term major muscle activity in daily life.

Another advantage of the invention is to provide a wearable device for posture correction and muscle training, which can be worn on the user's body to provide body posture correction and prevent bone adversely deformation.

Another advantage of the invention is to provide a wearable device for posture correction and muscle training, which provides resistance to core muscle groups, such as the muscle groups of the chest, abdomen, back, and legs, so that the muscles can be activated in a non-exercising state.

Another advantage of the invention is to provide a wearable device for posture correction and muscle training, which is suitable for being mounted on a chair or directly worn on the user's body, providing body posture correction and muscle exercise.

Another advantage of the invention is to provide a wearable device for posture correction and muscle training, which drives users to use their own muscles while standing or sitting, so that the user subconsciously uses muscle to maintain the physiological curvature of the spine to correct posture instead of forcibly tightening the user's body.

Another advantage of the invention is to provide a wearable device for posture correction and muscle training, wherein the wearable device comprises a wearable assembly and a resilience supporter, and the wearable assembly is directly arranged on the resilience supporter, thereby after the user's body attaching on the wearable assembly, the user exercises against the elastic force of the resilience supporter.

Another advantage of the invention is to provide a wearable device for posture correction and muscle training, which drives the user to apply force to resist the elastic tendency of the resilience supporter, so that the user can also use the muscles of waist, shoulders, chest or legs in daily life for avoiding the main muscle groups being in a relaxed state for a long time.

Another advantage of the invention is to provide a wearable device for posture correction and muscle training, wherein the wearable assembly comprises a waist fastener and a shoulder fastener, wherein the waist fastener and the shoulder fastener are arranged on the resilience supporter in a coherent manner, wherein the resilience supporter provides an upright elastic tendency to fit the waist and shoulders of the user's body to correct the posture.

Another advantage of the invention is to provide a wearable device for posture correction and muscle training, in which the user can activate major muscle groups through the wearable assembly, for example, activating the waist and shoulder muscle groups against the upright tendency of the resilience supporter.

Another advantage of the invention is to provide a wearable device for posture correction and muscle training, wherein the waist fastener is adapted to be mounted to the chair for the user to use in a sitting posture.

Another advantage of the invention is to provide a wearable device for posture correction and muscle training, which further comprises a leg fastener detachably arranged on the bottom end of the resilience supporter to fit the legs of the user's body to achieve the strength training to resist elasticity for the legs, waist and shoulders.

Another advantage of the invention is to provide a wearable device for posture correction and muscle training, which further comprises a neck fastener detachably arranged on the top end of the resilience supporter or the top end of the shoulder fastener, in order to fit the neck of the user's body to achieve the strength training to resist elasticity of the neck, waist, and shoulders.

Another advantage of the invention is to provide a wearable device for posture correction and muscle training, which further comprises a width fastener detachably arranged on the bottom end of the resilience supporter, providing adaptability according to different users to adjust size.

Another advantage of the invention is to provide a wearable device for posture correction and muscle training, which urges users to tighten their core muscles daily, and maintain a healthy bone posture through stretching of their own muscles to maintain a healthy body.

Another advantage of the invention is to provide a wearable device for posture correction and muscle training, in which the wearable assembly can be adjusted adaptively to meet the individual wearing positions of different users.

Additional advantages and features of the invention will become apparent from the description which follows, and may be realized by means of the instrumentalities and combinations particular point out in the appended claims.

According to the present invention, the foregoing and other objects and advantages are attained by a wearable device, for posture correction and muscle training of the user's body, comprising:

a wearable assembly and two resilience supporters, wherein the resilience supporter has an resilience tendency to be upright, wherein the wearable assembly is detachably arranged on the resilience supporter, wherein the wearable assembly is supported by the resilience supporter and is moved along with the resilience supporter, so that the user's body fits the wearable assembly and applies an force against the resilience supporter.

According to one embodiment of the present invention, the wearable assembly further comprises a waist fastener and two shoulder fasteners, which are detachably supported between the two resilience supporters, wherein the two shoulder fasteners are relatively arranged on the top ends of at least two resilience supporters, and the waist fastener is relatively arranged on the substantial bottom ends of the two resilience supporters for correspondingly fitting the waist and shoulders of the user's body.

According to one embodiment of the present invention, the waist fastener is vertically supported between the two resilience supporters, wherein the two shoulder fasteners are transversely supported on the two resilience supporters.

According to one embodiment of the present invention, the wearable assembly further comprises a leg fastener which is detachably supported on the bottom ends of the two resilience supporters.

According to one embodiment of the present invention, the wearable assembly further comprises a neck fastener which is detachably supported on the top ends of the two resilience supporters.

According to one embodiment of the present invention, the wearable assembly further comprises a width fastener which is detachably supported on the bottom ends of the two resilience supporters, so that the distance between the two bottom ends of the two resilience supporters is fixed and the distance between the two top ends of the two resilience supporter can be adjusted.

According to one embodiment of the present invention, the leg fastener and the width fastener of the wearable assembly are mutually interchangeably arranged on the two resilience supporters.

According to one embodiment of the present invention, the waist fastener of the wearable assembly further comprises two waist positioners and a connecting belt connected with the two waist positioners, wherein the two waist positioners are separately detachably arranged on the two resilience supporters, and the distance between the positions of the waist positioners of the two resilience supporters is adjustable by adjusting the length of the connecting belt. The waist fastener is suitable for being attached to the waist of the user's body, or suitable for being clamped to the chair back of a chair.

According to one embodiment of the present invention, each of the resilience supporters is a long and straight hollow tube.

According to one embodiment of the present invention, each of the resilience supporters is an elastic tube with at least two hollow channels therein.

Still further objects and advantages will become apparent from a consideration of the ensuing description and drawings.

These and other objectives, features, and advantages of the present invention will become apparent from the following detailed description, the accompanying drawings, and the appended claims.

### BRIEF DESCRIPTION OF THE DRAWINGS

FIGS. 1A and 1B are perspective views of a wearable device according to a preferred embodiment of the present invention.

FIG. 2 is an explosive view of the wearable device according to the above preferred embodiment of the present invention.

FIG. 3 is a perspective view of the wearable device according to the above preferred embodiment of the present invention, illustrating that the wearable device is mounted in a chair for the user in sitting posture.

FIG. 4 are front views of the wearable device according to the above preferred embodiment of the present invention, illustrating that the wearable device is adjustable transversally.

FIG. 5 are sectional views of the wearable device according to the above preferred embodiment of the present invention, illustrating that the wearable device is mounted in a chair for the user in sitting posture.

FIG. 6 is a perspective view of the wearable device according to the above preferred embodiment of the present invention, illustrating that a neck fastener of the wearable device is detachable arranged.

FIG. 7 is a perspective view of the wearable device according to the above preferred embodiment of the present invention, illustrating that a leg fastener of the wearable device is detachable arranged.

FIG. 8 are sectional views of the wearable device according to the above preferred embodiment of the present invention, illustrating that the leg fastener of the wearable device is arranged for the user in standing posture.

FIG. 9 is perspective views of the wearable device according to the above preferred embodiment of the present invention, illustrating an alternative mode of waist positioner for the user in standing posture.

### DETAILED DESCRIPTION OF THE PREFERRED EMBODIMENT

The following description is disclosed to enable any person skilled in the art to make and use the present invention. Preferred embodiments are provided in the following description only as examples and modifications will be apparent to those skilled in the art. The general principles defined in the following description would be applied to other embodiments, alternatives, modifications, equivalents, and applications without departing from the spirit and scope of the present invention.

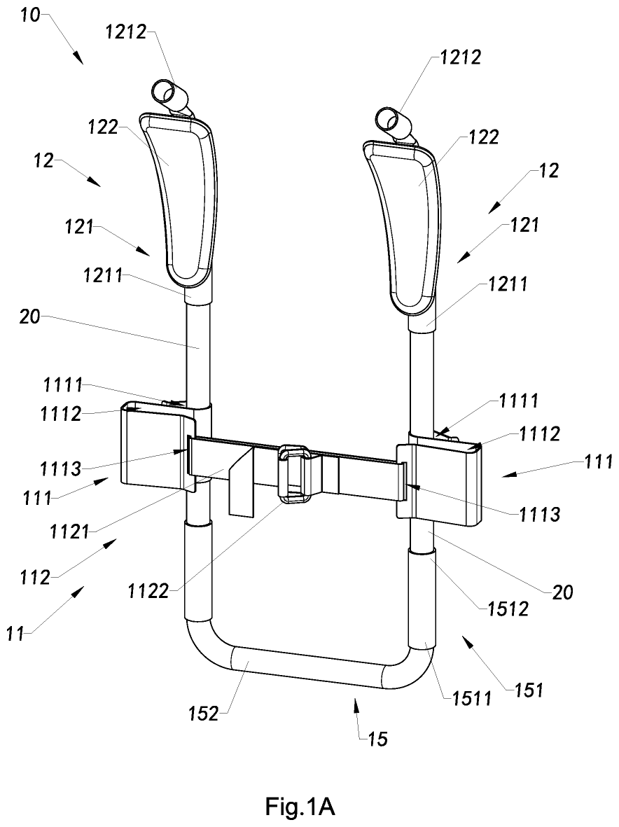

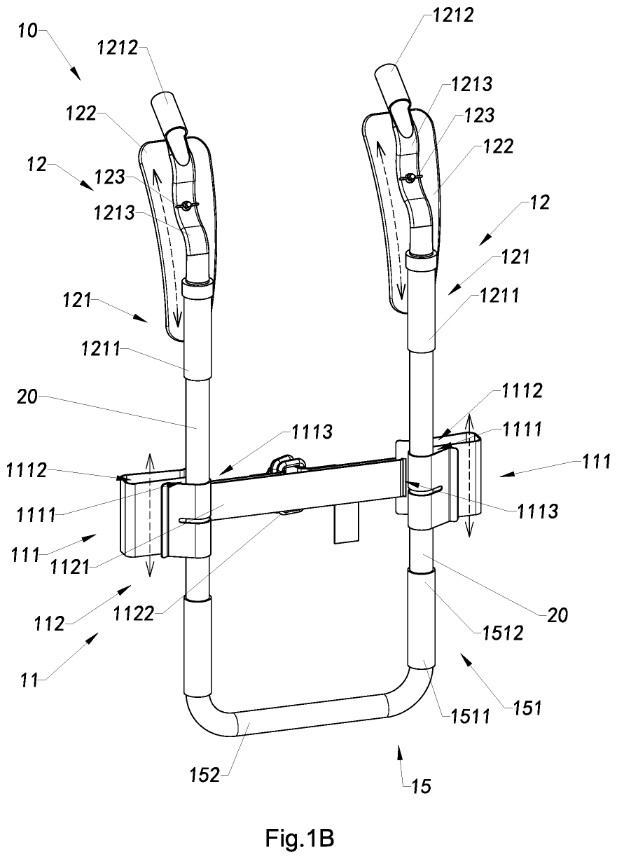

The present invention provides a preferred embodiment of a wearable device, as shown in FIGS. 1A to 8, which is suitable for a user 90 to use the wearable device for posture correction and muscle training in a sitting posture or a standing posture.

The wearable device comprises a wearable assembly 10 and two resilience supporters 20, and the wearable assembly 10 is detachably arranged on the resilience supporters 20. Preferably, the wearable assembly 10 is supported between two resilience supporters 20. The two of resilience supporters 20 are examples in the preferred embodiment. In some modified embodiments, the resilience supporters 20 may be manufactured in more than two. The wearable assembly 10 is suitable for being used to fitly attach on a user 90, and the user 90 is indirectly connected to the two resilience supporters 20. After the user 90 wears the wearable assembly 10 snugly, the two resilience supporters 20 are placed on two sides of the user 90 respectively.

It is worth to mention that the resilience supporter 20 has a tendency to remain upright. In other words, when the resilience supporter 20 is bent by applying a force, an elastic force for restoring straightness will be generated. The resilience supporter 20 provides the user 90 with the main core muscles to be trained counterforce. In an application, when the user 90 wears the wearable assembly 10 and bends down, it needs to oppose the elastic force of the resilience supporter 20, and needs to apply force from the core muscles of the abdomen or chest.

As shown in FIGS. 1A to 1B, the wearable assembly 10 provides at least two wearable parts corresponding to the user 90. The wearable assembly 10 further comprises a waist fastener 11 and two shoulder fasteners 12, wherein the two shoulder fasteners 12 are respectively arranged on the top ends of the two resilience supporters 20, wherein the waist fastener 11 is arranged on the two resilience supporters 20, preferably at a position relatively near the bottom ends of the two resilience supporters 20. The shoulder fastener 12 is extended relatively vertically from the top end of the resilience supporter 20, and the waist fastener 11 is relatively transversely supported between the two resilience supporters 20.

The wearable assembly 10 further comprises a width fastener 15 detachably arranged on the bottom ends of the two resilience supporters 20, so as to fix the length between the bottom ends of the two resilience supporters 20 with respect to the free top ends of the two resilience supporters 20. In one embodiment, the width fastener 15 is supported by two parallel resilience supporters 20, and the waist fastener 11 is vertically supported on the two resilience supporters 20 similarly to the width fastener 15, and the two shoulder fasteners 12 are respectively placed on the top ends of the two resilience supporters 20. As shown in FIGS. 1A to 3, the waist fastener 11 and the two resilience supporters 20 of the wearable device are substantially H-shaped, and the width fastener 15 and the two resilience supporters 20 are substantially U-shaped.

It is worth to mention that, in order to clearly explain the structure of the present invention, the top and bottom ends of the two resilience supporters 20 referred to in this embodiment respectively refer to the higher ends and the lower ends when the resilience supporters 20 are in a vertical using state.

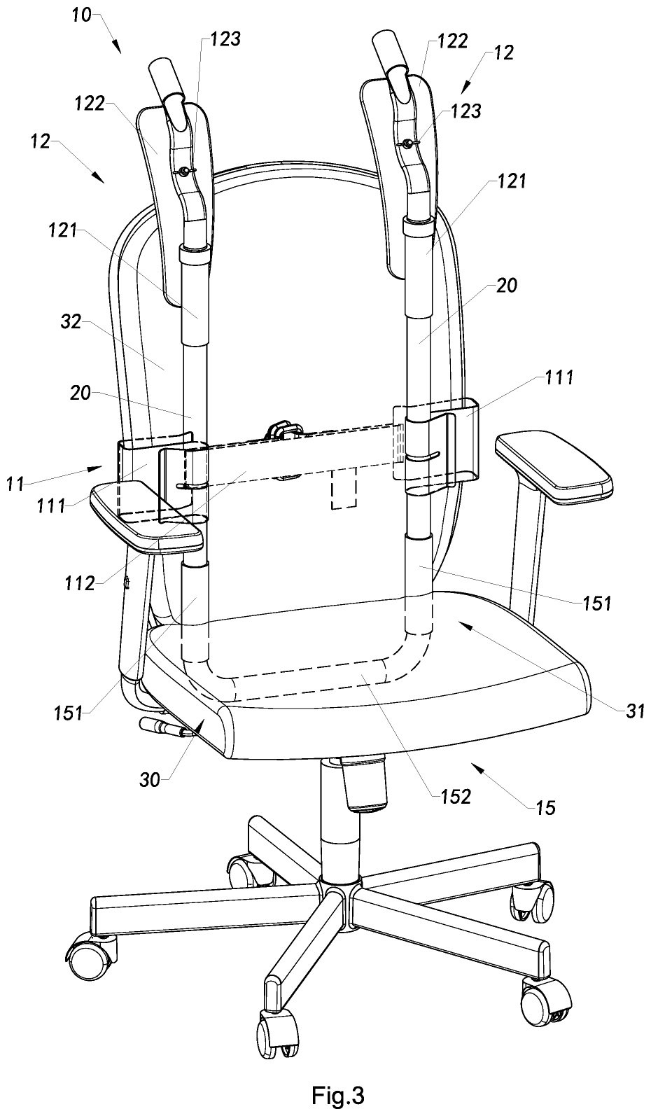

As shown in FIG. 3, each of the resilience supporters 20 is formed in a long strip shape with a hollow structure. The resilience supporter 20 has at least one hollow channel 200 axially penetrating therethrough. In the preferred embodiment, an elastic tube with the three hollow channels 200 are embodied. The resilience supporter 20 is preferably made of an elastic material, such as polymer plastic, rubber, or other polymer compounds.

Furthermore, the waist fastener 11 and the shoulder fastener 12 are arranged in a coherent manner on the resilience supporter 20. The resilience supporter 20 provides an upright elastic tendency. When the waist of the user 90 is attached on the waist fastener 11 and the shoulder is attached on the shoulder fastener 12, the major muscles can be trained against the elastic force of the resilience supporter 20. At the same time, the spine bones of the user 90 will be guided vertically, so that the posture of the waist and shoulders can be corrected.

More specifically, the waist fastener 11 further comprises two waist positioners 111 and a connecting belt 112. The two waist positioners 111 are detachably arranged on the two resilience supporters 20 respectively, wherein the connecting belt 112 is connected to the two waist positioners 111 such that the distance between the two resilience supporters 20 is determined by the length of the connecting belt 112. The connecting belt 112 is suitable for being worn on the waist of the user 90 so as to place the two resilience supporters 20 on left and right sides of the user 90 respectively. The circumference or length of the connecting belt 112 can be adjusted adaptively.

It is worth to mention that the position where the waist positioner 111 is arranged on the resilience supporter 20 can be adjusted. According to the body position of the user 90, the waist positioner 111 can be arranged at different position of the resilience supporter 20, for example, near the bottom end of the two resilience supporters 20 or near the middle position of the two resilience supporters 20. Alternatively, the two waist positioners 111 are respectively hold on the two resilience supporters 20. Preferably, the two waist positioners 111 are respectively engaged with the two resilience supporters 20. In more feasible embodiments, the two waist positioners 111 are respectively sheathed on the outer periphery of the two resilience supporters 20, so as to be slidable along the resilience supporters 20.

As shown in FIGS. 1A to 3, the waist positioner 111 in the preferred embodiment further comprises a supporter connector 1111, a chair connector 1112, and a connecting belt connector 1113, wherein the supporter connector 1111 is extended from the chair connector 1112 to the connecting belt connector 1113. The supporter connector 1111 is detachably arranged on the outer periphery of the resilience supporter 20. The connecting belt connector 1113 is receiving the connecting belt 112 therein. In other words, the waist positioner 111 can not only be arranged on the resilience supporter 20, but also can holding the connecting belt 112 inside. By changing the engagement position of the supporter connector 1111 of the waist positioner 111 relative to the resilience supporter 20, the height position of the connecting belt 112 relative to the resilience supporter 20 can be adjusted. That is to say, according to the needs of the user 90, the position where the supporter connector 1111 is engaged with the resilience supporter 20 can be adjusted, and the connecting belt 112 can be personalized.

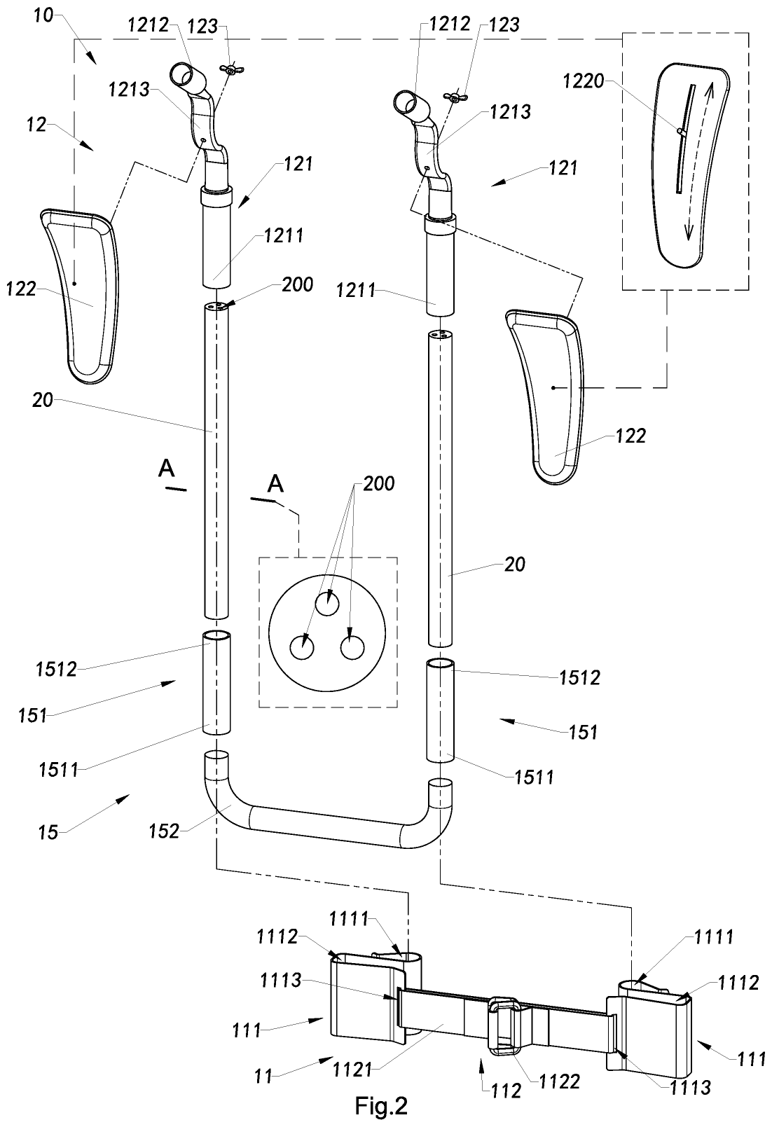

In the preferred embodiment, the supporter connector 1111 and the chair connector 1112 are respectively U-shaped clips. The supporter connector 1111 is bent to form a cavity for engaging the resilience supporter 20, and the chair connector 1112 is bent to form a cavity for engaging a chair 30. The openings of the supporter connector 1111 and the chair connector 1112 are oppositely arranged. As shown in FIG. 2, the supporter connector 1111, the chair connector 1112 and the connecting belt connector 1113 are integrally formed into two-curved clips. The connecting belt connector 1113 is an opening at one end of the clip for the connecting belt 112 passing through.

Furthermore, the connecting belt 112 further comprises a belt body 1121 and a set of connecting buckles 1122. The belt body 1121 is passed through the connecting belt connectors 1113 of the two waist positioners 111 to be fixed by the connecting buckle 1122, and then surrounds the two resilience supporters 20 in a loop for the user 90 wearing. The circumference of the belt body 1121 can be adjusted by the connecting buckle 1122. Then, adjusting the length of the connecting belt 112 between the two resilience supporters 20 can change the distance between the two waist positioners 111, thereby adjusting the distance between two resilience supporters 20 by adjusting the clamped waist positioner 111.

As shown in FIG. 3, the waist positioner 111 is mounted to a chair back 32 of the chair 30 is illustrated. Both sides of the chair back 32 are respectively clamped by the chair connectors 1112 of the waist positioner 111, and the two resilience supporters 20 are clamped by the supporter connectors 1111 of the waist positioner 111 holding on the front side of the chair back 32, and the belt body 1121 of the connecting belt 112 is placed on the back side of the chair back 32 by the connecting belt connector 1113 of the waist positioner 111. That is, the two waist positioners 111 are clamped and fixed on the left and right sides of the chair back 32, and the belt body 1121 of the connecting belt 112 and the resilience supporter 20 are attached to the chair back 32 on the front and rear sides of the chair back 32, so that the resilience supporter 20 is vertically placed on the front side of the chair back 32.

As shown in FIGS. 1A to 3, each of the shoulder fasteners 12 further comprises a shoulder extending frame 121 and a shoulder holder 122, wherein the shoulder extending frame 121 is detachably extended from the top end of the resilience supporter 20, and the shoulder holder 122 is detachably arranged on the shoulder extending frame 121. The shoulder extending frame 121 is preferably sheathed on the top end of the resilience supporter 20. A bottom end 1211 of the shoulder extending frame 121 is cylindrical, and a top end 1212 is bent and is extended from the bottom end 1211 through an elastic middle portion 1213. The top end 1212 of the shoulder extending frame 121 is also formed into a cylindrical shape in the preferred embodiment. The shoulder holder 122 is detachably arranged on the elastic middle portion 1213 of the shoulder extending frame 121. The shoulder holder 122 is a sheet with an arched surface, which is suitable for attaching on the front of the shoulder of the user 90.

The shoulder extending frame 121 not only is extended upward from the resilience supporter 20, but also the elastic middle portion 1213 of the shoulder extending frame 121 is extended in the direction of the chair connector 1112 of the waist positioner 111. In other words, the shoulder extending frame 121 has an elastic tendency toward the chair connector 1112 of the waist positioner 111. When the front of the shoulder of the user 90 fits the shoulder holder 122 and squeezes the elastic middle portion 1213 of the shoulder extending frame 121 forwardly, the elastic middle portion 1213 of the shoulder extending frame 121 will give the user 90 pushing force, to assist the user 90 to correct posture of the spine, and urges the user 90 to train the core muscles of the shoulder.

Furthermore, the shoulder fastener 12 further comprises a sliding member 1220, and the sliding member 1220 is slidably provided in the shoulder holder 122. The sliding member 1220 of the shoulder holder 122 is adapted to be fixedly arranged on the elastic middle portion 1213 of the shoulder extending frame 121. In the preferred embodiment, the sliding member 1220 is a protrusion of the shoulder holder 122, and the elastic middle portion 1213 of the shoulder extending frame 121 has a mounting hole corresponding to the sliding member 1220. After the sliding member 1220 is inserted into the mounting hole of the elastic middle portion 1213 of the shoulder extending frame 121, the shoulder holder 122 is assembled. By moving the shoulder holder 122 up and down, the position of the elastic middle portion 1213 relative to the shoulder extending frame 121 can be adjusted to match the posture of the user 90.

The shoulder fastener 12 further comprises an adjusting buckle 123 that is matched and adaptably connected to the sliding member 1220. The sliding member 1220 is mounted on the elastic middle portion 1213 of the shoulder extending frame 121. After the relative position of the shoulder holder 122 is determined, the adjusting buckle 123 is connected to the sliding member 1220 to determine the relative position between the sliding member 1220 and the shoulder holder 122. In the preferred embodiment, the adjusting buckle 123 is a nut, and the sliding member 1220 has a corresponding thread. After the sliding member 1220 is mounted from one side, the adjusting buckle 123 locks the relative position of the shoulder holder 122 from the other side of the elastic middle portion 1213 of the shoulder extending frame 121.

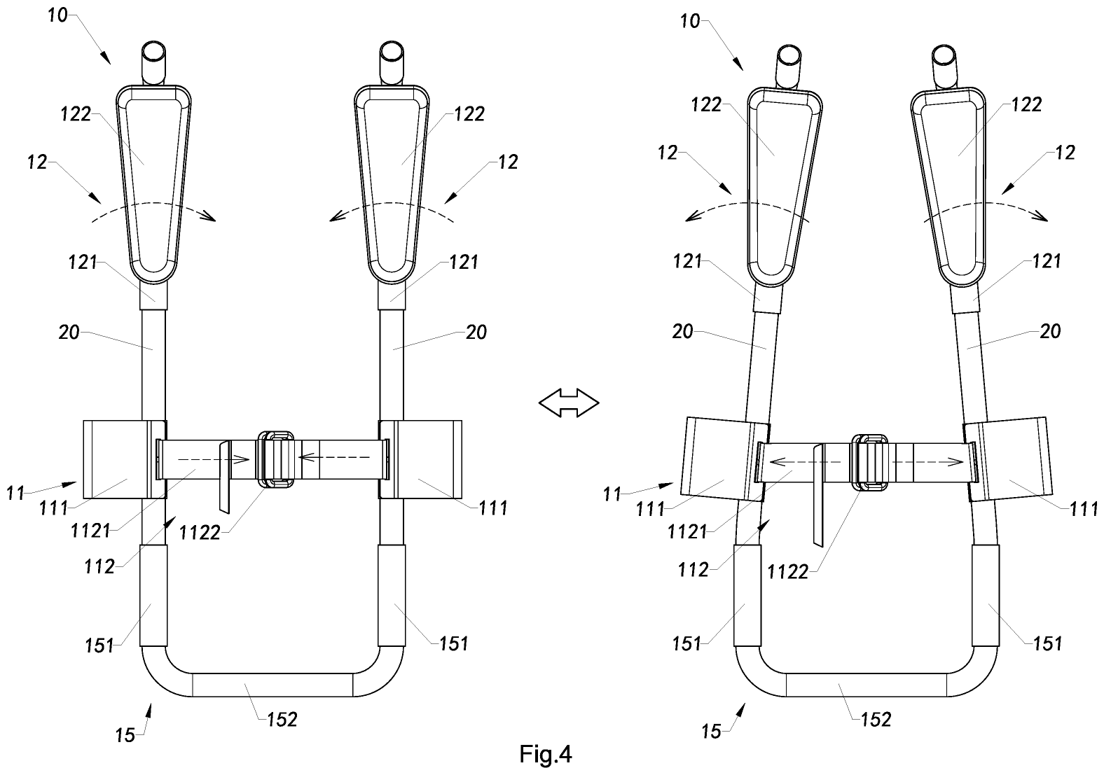

As shown in FIG. 4, the width fastener 15 further comprises two width extending frames 151 and a base frame 152 extending between the two width extending frames 151. Each width extending frame 151 is detachably connected to the bottom end of the resilience supporter 20 respectively. Specifically, in the preferred embodiment, a top end 1512 of the width extending frame 151 is vertically sleeved on the bottom end of the resilience supporter 20, and a bottom end 1511 of the width extending frame 151 is transversely connected to the base frame 152. The base frame 152 has a fixed length and serves as a fulcrum of the two resilience supporters 20. By adjusting the length of the belt body 1121 of the connecting belt 112 between the two resilience supporters 20, the distance between the two shoulder fasteners 12 can be changed, so that the distance between the two shoulder holders 122 can be adjusted so that the user 90 can be attached on the shoulder holder 122 for wearing.

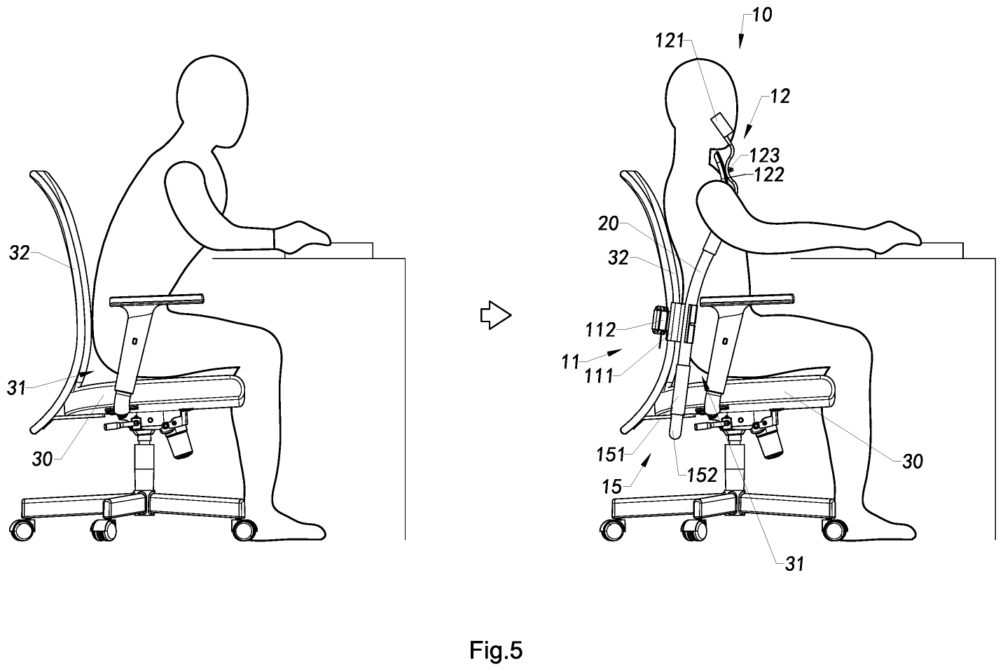

It is worth to mention that the wearable device, shown in FIG. 3, is mounted to the chair 30 in a combined state. In specific use, the user 90 can adjust the length of the belt body 1121 of the connecting belt 112 to adjust the distance between the two shoulder holders 122 to adapt to the width of the shoulders of the body. The sliding member 1220 can also be adjusted to adjust the height of the shoulder holder 122 to adapt to the height of the shoulders of the body. As shown in FIG. 5, the user 90 is sitting at the desk on a chair seat 31 of the chair 30. Without the wearable device, the user is likely to be completely relaxed, the mayor muscles are tightened, and the spine is unhealthy bent forward. However, after the wearable device is mounted on the chair 30 in advance, the shoulders of the user 90 in a sitting position wears on the shoulder holder 122, and the resilience supporter 20 has a backward elastic force, and not only the spine of user 90 needs to be kept upright, and the muscles need to resist the elastic force of the resilience supporter 20, so that the muscle groups of the user 90, especially the abdominal and chest muscles, are stretched to apply force. Therefore, the user 90, in a sitting posture, can use the wearable device to exert force from the shoulders to train the body, so that the muscle strength can be activated even in the non-moving state. In addition, the wearable device does not occupy the space of the chair seat 31 and does not reduce the comfort of the chair 30.

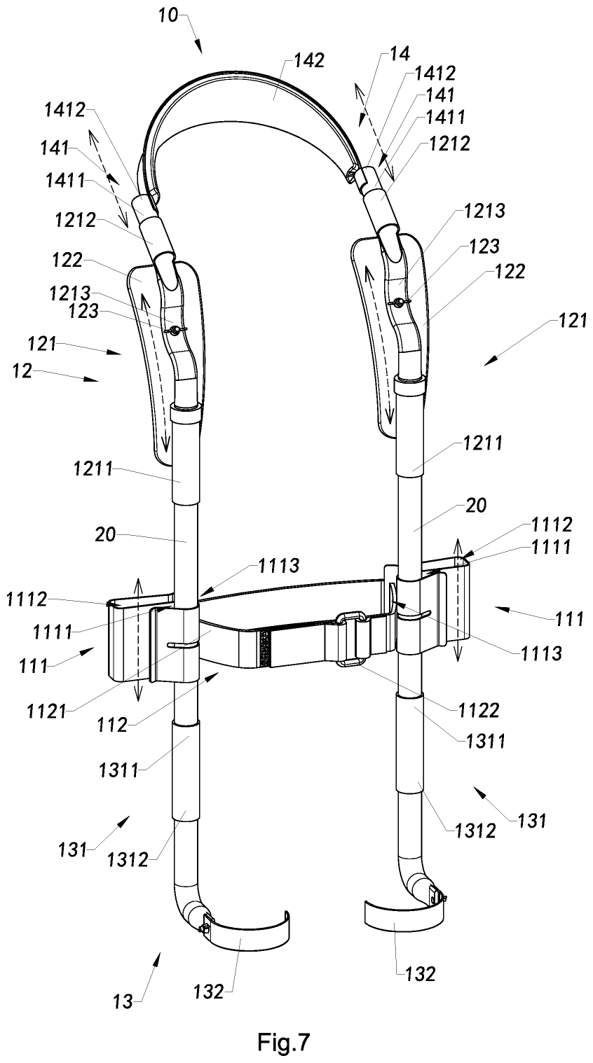

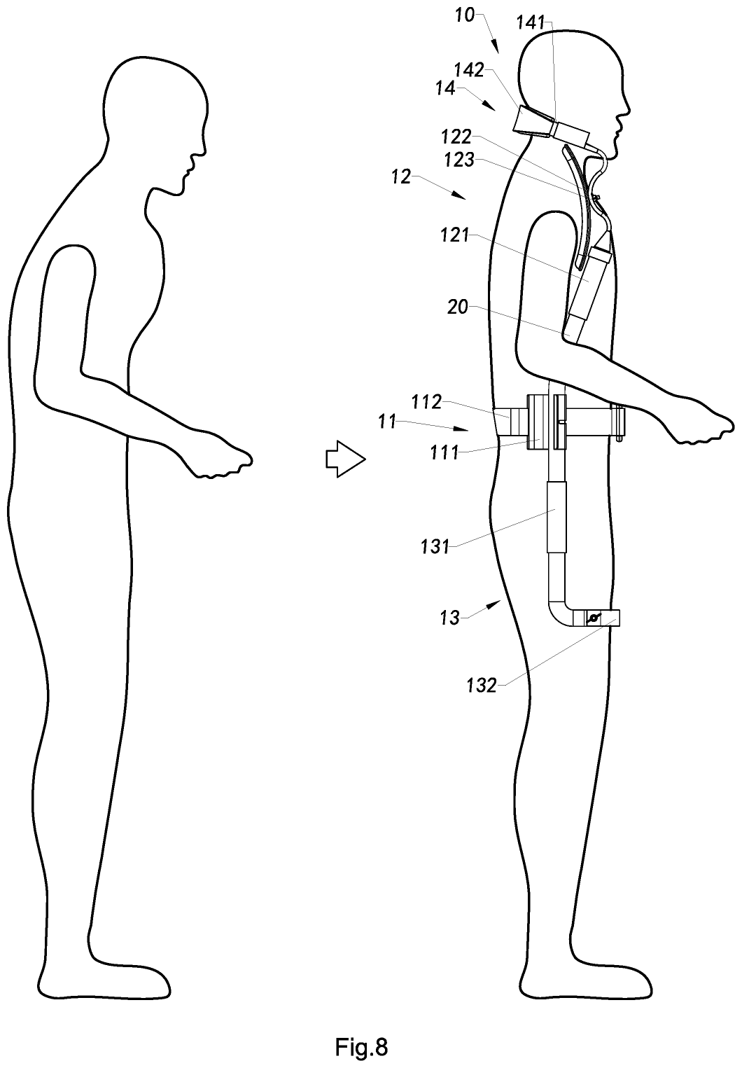

As shown in FIGS. 7 and 8, the wearable assembly 10 further comprises two leg fasteners 13 which are respectively detachably arranged on the bottom ends of the two resilience supporters 20. Each of the leg fasteners 13 is extended downward from the resilience supporter 20 for the legs of the user 90 to wear. In other words, the legs of the user 90 attach on the leg fastener 13, and the leg muscles of the user 90 are trained by applying a force to the leg fastener 13 to resist the elastic force of the resilience supporter 20.

Furthermore, each of the leg fasteners 13 further comprises a leg extending frame 131 and a leg binding holder 132 extending transversely from the leg extending frame 131. A top end 1312 of the leg extending frame 131 is extended vertically downward from the resilience supporter 20, and a bottom end 1312 of the leg extending frame 131 is extended vertically to the leg binding holder 132. The leg binding holder 132 is in the shape of a sheet with an arched surface to fit the shape of the user 90 leg. The top end 1312 of the leg extending frame 131 is sheathed on the bottom end of the resilience supporter 20 so that the legs of the user 90 can apply force the leg binding holder 132 to resist the elastic force of the resilience supporter 20. In an alternative embodiment, the leg binding holder 132 is axially connected to the bottom end 1312 of the leg extending frame 131.

It is worth to mention that the leg extending frame 131 of the leg fastener 13 and the width extending frame 151 are interchangeably connected to the resilience supporter 20. In other words, the wearable device can be used with a fixed width, for example, attached to the chair 30, or can be worn on the legs for use, such as standing or walking. In particular, as shown in FIG. 8, when the user 90 wears the waist fastener 11, the shoulder fastener 12, and the leg fastener 13 of the wearable assembly 10, the three major muscles of the waist, the shoulders, and the leg of the user 90 are stressed and can be fully trained.

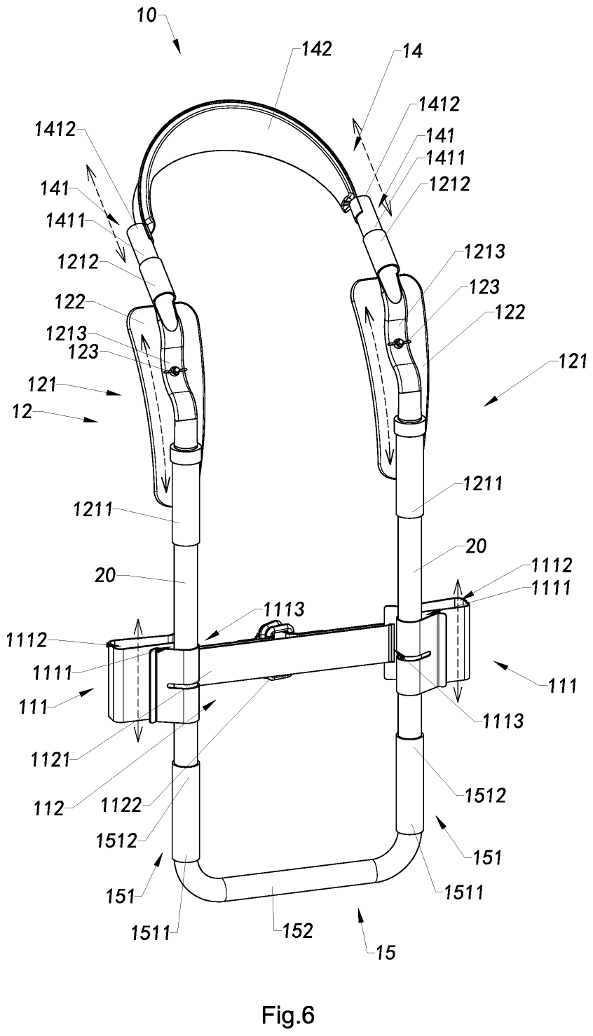

As shown in FIGS. 6 to 8, the wearable assembly 10 further comprises a neck fastener 14 which is detachably arranged on the top end 1212 of the shoulder extending frame 121 of the two shoulder fasteners 12. The neck fastener 14 is adapted to attach on the back neck of the user 90 and provide support for the neck or head of the user 90.

The neck fastener 14 further comprises two neck extending frames 141 and a neck holder 142 curvedly extended between the two neck extending frames 141. Each neck extending frame 141 is detachably arranged on the top end 1212 of the shoulder extending frame 121. In the preferred embodiment, the top end 1212 of the shoulder extending frame 121 is shaped in tube, and the neck extending frame 141 is cylindrical for accessing the top end 1212 of the shoulder extending frame 121. The outer diameter of a bottom end 1411 of the neck extending frame 141 is designed to correspond to the inner diameter of the top end 1212 of the shoulder extending frame 121. The top ends 1412 of the two neck extending frames 141 serve as arch supporting points of the neck holder 142. By adjusting the distance between the bottom end 1411 of the neck extending frame 141 and the top end 1212 of the shoulder extending frame 121, the neck holder 142 can be worn on the neck of the user 90 adaptively.

In another embodiment, the neck extending frame 141 of the neck fastener 14 is detachably arranged on the top end of the resilience supporter 20 without the shoulder fastener 12 being indirectly assembled.

The neck fastener 14, the shoulder fastener 12, the waist fastener 11, and the leg fastener 13 of the wearable assembly 10 are sequentially arranged from the top end to the bottom end of the resilience supporter 20 in a coherent manner. The resilience supporter provides an upright elastic tendency to attach to the neck, shoulders, waist and legs of the user's body to correct the posture, and to urge the daily training core muscles to ensure healthy stretching of muscles and bone posture, so as to maintain a healthy state.

In the preferred embodiment, the top end 1212 of the shoulder extending frame 121 is matched and connected with the bottom end 1411 of the neck extending frame 141, and the bottom end 1211 of the shoulder extending frame 121 is matched and connected with the resilience supporter 20, and the resilience supporter 20 is matched and connected with the top end 1512 of the width extending frame 151, and the resilience supporter 20 is matched and connected with the top end 1311 of the leg extending frame 13. The preferred connection method is muff-coupling connection. Of course, in other embodiments, it can be implemented as a threaded connection, a slot connection, and so on.

In particular, the arched arcs of the neck holder 142, the shoulder holder 142 and the leg binding holder 132 are adapted to the design in manual engineering.

As shown in FIG. 8, when the user 90 is in a standing position, if there is no such wearable device, the user is likely to be in a completely relaxed state, which is prone to hunchback. When the user 90 wears the waist fastener 11, the shoulder fastener 12, the neck fastener 14 and the leg fastener 13 of the wearable assembly 10, the resilience supporter 20 has a tendency to be back upright, the user 90 resists the elastic force of the resilience supporter 20 by applying force to the shoulder holder 122, the neck holder 142, and the leg binding holder 132. It is worth to mention that the resilience supporter 20 is a bar with strong elastic restoring force. The user 90 not only needs to keep the spine upright, but also the muscles need to resist the elastic force of the resilience supporter 20, so that the core muscles of the user 90, especially the abdominal and chest muscles, can be stretched backward.

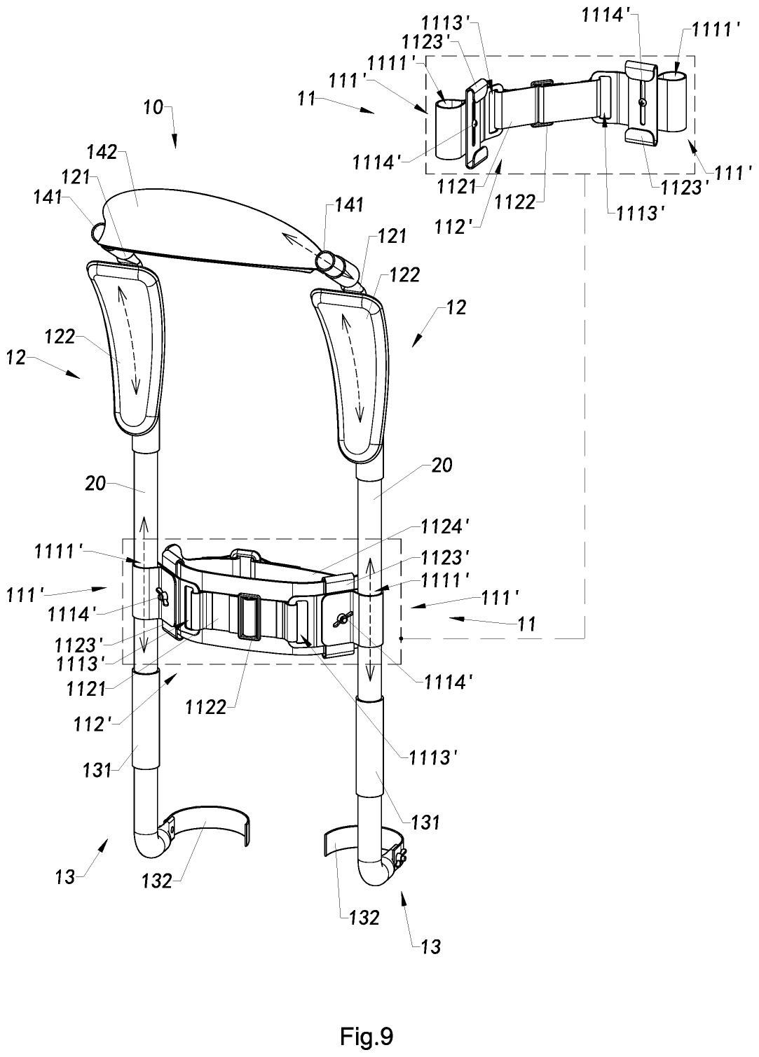

An alternative mode of the present invention is shown in FIG. 9, and the wearable assembly 10 is the similar as in the above mentioned embodiment and will not be repeated in the following disclosure. The difference is that the wearable assembly 10 comprises a waist positioner 111′ and an adjusting belt 112′, wherein the waist positioner 111′ further comprises a supporter connector 1111′ and an adjusting belt connector 1113′. That is to say, the chair connector 1112 is not provided in this embodiment. And the adjusting belt 112′ comprises a belt body 1121, a set of connecting buckles 1122, a belt holder 1123′ and a binding belt 1124′. The supporter connector 1111′ is similar to the above embodiment to adjustably hold on the resilience supporter 20, and the adjusting belt 112′ is similar to the above embodiment to adjustably hold on the belt body 1121. More particularly, the binding belt 1124′ is suitable for binding to the waist of the user, and preferably the binding belt 1124′ is wider than the belt body 1121. The belt holder 1123′ is fixed to the waist positioner 111′ to accommodate and fix the binding belt 1124′. Preferably, there are multiple belt holder 1123′. In this alternative mode, the belt holder 1123′ is placed in the supporter connector 111′ in a U-shape, so that the binding belt 1124′ is placed in the opening of the belt holder 1123′. Furthermore, the belt holder 1123′ is further fixed to the waist positioner 111′ by an adjustable turnbuckle 1114′. In other words, the waist fastener 111′ further has a groove, which is suitable for fixing the turnbuckle 1114′ from one side to the belt holder 1123′ on the other side. In more alternative embodiments, the binding belt 1124′ can be implemented as an conventional waistband, and the belt holder 1123′ is used to clamp the binding belt 1124′ so as to be fixed relative to the waist positioner 111′. It is worth to mention that the wearable assembly 10 of the wearable device does not tightly bind the user 90, but provides force points that fits the main muscle group, ensuring long-term muscle activity in daily life, and can provide portable posture correction, which makes the user 90 subconsciously use muscle to maintain the physiological curvature and correct posture of the spine, instead of forcibly tightening the user's body. The wearable assembly 10 is not easy to fall after being worn, assists the bones to stay upright, provides correction of body posture, and prevents bad bone deformation.

The wearable assembly 10 can be assembled onto the resilience supporter 20 according to the needs of the user 90, which is convenient for various applications.

One skilled in the art will understand that the embodiment of the present invention as shown in the drawings and described above is exemplary only and not intended to be limiting.

It will thus be seen that the objects of the present invention have been fully and effectively accomplished. The embodiments have been shown and described for the purposes of illustrating the functional and structural principles of the present invention and is subject to change without departure from such principles. Therefore, this invention comprises all modifications encompassed within the spirit and scope of the following claims.

What is claimed is:

1. A wearable device, for posture correction and muscle training, comprising:
a wearable assembly; and
two resilience supporters, wherein said two resilience supporters have an resilience tendency to be upright, wherein said wearable assembly is detachably arranged on said two resilience supporters, wherein said wearable assembly comprises a waist fastener and two shoulder fasteners, wherein said waist fastener is detachably supported between said two resilience supporters on two substantial bottom ends of said two resilience supporters relatively, wherein said two shoulder fasteners are arranged to two top ends of said two resilience supporters relatively, so that said wearable assembly are correspondingly fitted to waist and shoulders of a user's body.

2. The wearable device, as recited in claim 1, wherein said wearable assembly is supported by said two resilience supporters and is driven to move along with a movement of said two resilience supporters, so that the user's body applies force against said two resilience supporters by fitting said wearable assembly.

3. The wearable device, as recited in claim 2, wherein said two shoulder fasteners are vertically supported on said two resilience supporters, wherein said waist fastener is transversely supported between said two resilience supporters.

4. The wearable device, as recited in claim 3, wherein said waist fastener of said wearable assembly further comprises two waist positioners and a connecting belt connected to said two waist positioners, wherein said two waist positioners are detachably arranged on said two resilience supporters respectively, so that a distance between positions of said waist fasteners on said two resilience supporters is adjusted by adjusting a length of said connecting belt.

5. The wearable device, as recited in claim 4, wherein said two waist positioners are respectively hold on said two resilience supporters.

6. The wearable device, as recited in claim 4, wherein said two waist positioners are respectively clamped to said two resilience supporters.

7. The wearable device, as recited in claim 4, wherein said waist positioner further comprises a supporter connector, a chair connector and a connecting belt connector, wherein said supporter connector is extended from said chair clip to said connecting belt connector, wherein said supporter connector is detachably arranged to hold on an outer periphery of said resilience supporter, and said connecting belt is received to be hold in said connecting belt connector.

8. The wearable device, as recited in claim 7, wherein said supporter connector is bent to form a cavity for engaging said resilience supporter, and said chair connector is bent to form a cavity for engaging with a chair.

9. The wearable device, as recited in claim 7, wherein said supporter connector and said chair connector are respectively U-shaped, wherein an openings of said supporter connector and said chair connector are faced oppositely.

10. The wearable device, as recited in claim 7, wherein said supporter connector, said chair connector, and said connecting belt connector are integrally formed into a two-curved clip, wherein said connecting belt is formed into an opening at one end of said two-curved clip for said connecting belt passing through.

11. The wearable device, as recited in claim 4, wherein each of said shoulder fasteners further comprises a shoulder extending frame and a shoulder holder, wherein said shoulder extending frame is detachably extended from a top end of said resilience supporter, wherein said shoulder holder is detachably arranged on said shoulder extending frame, and said shoulder holder is a sheet with an arched surface.

12. The wearable device, as recited in claim 11, wherein said shoulder extending frame is sheathed on said top end of said resilience supporter, wherein a bottom end of said shoulder extending frame is cylindrically shaped.

13. The wearable device, as recited in claim 11, wherein said wearable assembly further comprises a neck fastener which is detachably supported on two top ends of said two shoulder fasteners.

14. The wearable device, as recited in claim 13, wherein said neck fastener further comprises two neck extending frames and a neck holder curvedly extended between said two neck extending frames, wherein each of said neck extending frames is detachably arranged on a top end of said shoulder extending frames.

15. The wearable device, as recited in claim 13, wherein a top end of said shoulder extending frame is cylindrical, wherein said neck extending frame is cylindrical for connecting to said top end of said shoulder extending frame, wherein an outer diameter of a bottom end of said neck extending frame corresponds to an inner diameter of said top end of said shoulder extending frame, so that a distance between said bottom end of said neck extending frame and said top end of said shoulder extending frame is adjustable.

16. The wearable device, as recited in claim 4, wherein said wearable assembly further comprises a width fastener which is detachably supported on two bottom ends of said two resilience supporters, so that a distance between said bottom ends of said two resilience supporters is fixed and a distance between said top ends of said two resilience supporters adjustable.

17. The wearable device, as recited in claim 4, wherein said wearable assembly further comprises a leg fastener which is detachably supported on said bottom ends of said two resilience supporters.

18. The wearable device, as recited in claim 17, wherein each of said leg fasteners further comprises a leg extending frame and a leg binding holder is vertically extended from said leg extending frame, wherein a top end of said leg extending frame is extended vertically downward from said resilience supporter, wherein a bottom end of said leg extending frame is extended transversely to said leg binding holder, wherein said leg binding holder has an arch shape.

19. The wearable device of claim 18, wherein said top end of said leg extending frame is sheathed at said bottom end of said resilience supporter.

20. The wearable device, as recited in claim 17, wherein said wearable assembly further comprises a width fastener which is detachably supported on two bottom ends of said two resilience supporters, so that a distance between said bottom ends of said two resilience supporters is fixed and a distance between said top ends of said two resilience supporters is adjustable.

21. The wearable device, as recited in claim 20, wherein said leg fastener and said width fastener of said wearable assembly are mutually interchangeably arranged on said two resilience supporters.

22. The wearable device, as recited in claim 1, wherein each of said resilience supporters is a long and straight hollow tube.

23. The wearable device, as recited in claim 1, wherein each of said resilience supporters is an elastic tube with at least two hollow channels therein.
